(19) United States
(12) Patent Application Publication
(10) Pub. No.: US 2006/0233845 A1
(43) Pub. Date: Oct. 19, 2006

(54) Micro/nanoparticle obtained from lipid-containing marine organisms for use in pharmaceutics and cosmetics

(76) Inventors: Gerold Lukowski, Greifswald (DE); Wolf-Dieter Juelich, Greifswald (DE); Ulrike Lindequist, Greifswald (DE); Sabine Mundt, Stralsund (DE)

Correspondence Address: JORDAN AND HAMBURG LLP, 122 EAST 42ND STREET, SUITE 4000, NEW YORK, NY, 10168, US

(21) Appl. No.: 10/507,061

(22) PCT Filed: Feb. 28, 2003
(86) PCT No.: PCT/DE03/00747

§ 371 Date: Aug. 3, 2005

(30) Foreign Application Priority Data
Feb. 28, 2002 (DE) 102 08 949.3
Aug. 24, 2002 (DE) 102 08 364.9

(51) Int. Cl. A61K 8/36; C12P 7/64; A61K 8/49

(52) U.S. Cl. 424/401; 435/134; 977/926

(57) ABSTRACT

The invention relates to pharmaceutically or cosmetically active agents, which are obtained by converting biomasses consisting of lipid-containing marine organisms into microparticles and nanoparticles and which preferably have an average diameter of 10 nm 10 μm. Possible fields of application of these agents include the field of medicine, the production of cosmetics or the production of foodstuffs and, in particular, the agents are used for the prophylaxis of nosocomial infections, accelerating cell growth and for inhibiting staphylococci.

The invention relates to pharmaceutically or cosmetically active agents, which are obtained by converting biomasses consisting of lipid-containing marine organisms into microparticles and nanoparticles and which preferably have an average diameter of 10 nm to 10 μm. Possible fields of application of these agents include the field of medicine, the production of cosmetics or the production of foodstuffs.

Marine organisms, in particular microalgae and macroalgae, marine fungi (thraustochytrids) and marine bacteria contain high concentrations of lipids and display a specific lipid composition (Lindequist, U. and Th. Schweder: Marine Biotechnology, in: Biotechnology. 2. ed., Ed. H.-J. Rehm Wiley-VCH Weinheim 2001, pp. 442-473).

Accordingly, e.g. microalgae can store up to 70% of the assimilated carbon in the form of energetically efficient lipids when being submitted to growth limiting conditions, in particular to N-limitation. The concentration and composition of these lipids can be affected by the growth conditions. Whereas higher plants predominantly produce lipids containing relatively short-chained fatty acids (C12-C18) and a low number of double bonds (up to C18:3), the variability of fatty acids produced by the marine organisms is incomparably higher. Algae synthesise many polyunsaturated C16-C22 fatty acids, e.g. linoleic acid (C18:2), linolenic acid (C18:3), arachidonic acid (C20:4), eicosapentaenoic acid (EPA, C20:5) and docosahexaenoic acid (DHA, C22:6) (Pohl, P. and F. Zurheide: Fatty acids and lipids of marine algae and the control of their biosynthesis by environmental factors. In: Marine Algae in Pharmaceutical Science. Ed. H. A. Hoppe, T. Levring, Y. Tanaka, Walter de Gruyter New York, 1979, pp. 473-523; Roessler, P. G.: Environmental control of glycerolipid metabolism in microalgae: commercial implications and future research directions. J. Phycology 26, 393-399, 1990; Puls, O.: Biotechnology with cyanobacteria and microalgae, in: Biotechnology, 2. ed., Ed. H.-J. Rehm Wiley-VCH Weinheim 2001, pp. 105-136, Servel, M. O., C. Claire, A. Derrien, L. Ciffard, Y. DeRoeck-Holtzhauer: Fatty acid composition of some marine microalgae. Phytochemistry 36, 691-693, 1994). The heterotrophic microalga Cryptecodinium cohnii can produce up to 13% of its cellular mass in the form of lipids containing 36-43% DHA (DeSwaaf, M. E., DeRijk, T. C., Eggink, G., Sijtsma, L.: Optimisation of docosahexaenoic acid production in batch cultivations by Crypthecodinium cohnii. J. Biotech. 70, 185-192, 1999).

Thraustochytrids are fungal protists, which produce up to 50% of their lipids in the form of DHA and deposit these lipids as oil drops in the cytoplasm (Lewis, T. E., Nichols, P. D., McMeekin, T. A.: The biotechnological potential of thraustochytrids, Mar. Biotechnol. 1, 580-587 1999).

Marine bacteria in contrast incorporate their polyunsaturated fatty acids in membrane phospholipids. The concentration of EPA in the total lipids of Colwellia psychrerythrea is 6-7% (Bowman, J. P., Gosink, J. J., McCammon, S. A., Lewis, T. T., Nichols, D. S., Skerratt, J. H., Rea, S. M., McMeekin, T. A.: Colwellia demingae sp. nov., Colwellia hornerae sp. nov., Colwellia rossensis sp. nov. and Colwellia psychrotropica sp. nov.: psychrophilic Antarctic species with the ability to synthesise docosahexaenoic acid (22:6 n-3), Int. J. Syst. Bacteriol. 48, 1171-1180, 1997).

Linoleic acid (Z,Z-9,12-octadecadienoic acid) and linolenic acid (Z,Z,Z-9,12,15-octadecatrienoic acid) as essential fatty acids are irreplaceable for human nutrition. Arachidonic acid (5,8,11,14-eicosatetraenoic acid) is the precursor of the physiologically important eicosanoids. EPA and DHA (omega-3 fatty acids) are not only crucial in early childhood brain development, but also are of prophylactic/therapeutic benefit in cardiovascular diseases, rheumatic diseases, chronically inflammatory bowel diseases, neurodermatitis, psoriasis and allergies. It was e.g. demonstrated, that the mortality rate of patients after having suffered from a cardiac infarction was significantly reduced by the daily ingestion of omega-3 fatty acids (GISSI study). Of particular interest are structurally exceptional fatty acids, which exhibit further biological properties. The hydroxy fatty acids coriolic acid (13-HODE Z,E) and dimorphecolic acid (9-HODE E,Z) being isolated from an Oscillatoria species, similar to the linolenic acid are characterised by an antibacterial activity (Kreitlow, S.: Dissertation Greifswald 2000).

In addition to their fatty acid content, the marine organisms also are of interest in the fields of nutrition, cosmetics and medicine for reason of their plenitude of proteins, vitamins and mineral substances (Lindequist, U. and Th. Schweder: Marine Biotechnology, in: Biotechnology. 2. ed., Ed. H.-J. Rehm Wiley-VCH Weinheim 2001, pp. 442473; Puls, O.: Biotechnology with cyanobacteria and microalgae, in: Biotechnology, 2. ed., Ed. H.-J. Rehm Wiley-VCH Weinheim 2001, pp. 105-136, Becker, Microalgae: Biotechnology and Microbiology, Cambridge, 1994; Köhler, Kurth, Pulz, Spirulina platensis—an microalgae additiv for cosmetics, Biotechnology for Microalgae, 1997).

The production and use of extracts obtained from algae and the employment of algae biomass in the fields of nutrition, cosmetics and medicine is known from patent literature (e.g. U.S. Pat. No. 4,320,050 A, IL 59766, DE 100 59 107 A1). Also described were specific combinations of fibrillin with blue algae extracts in cosmetics and medicine (WO 01/07006 A1). Other patents relate to the production of polyunsaturated fatty acids by marine organisms (e.g. Barclay, W. R. (1992) Process for the heterotrophic production of microbial oils with high concentrations of omega-3 highly unsaturated fatty acids, U.S. Pat. No. 5,130,242 A; Barclay, W. R. (1994) Process for growing Thraustochytrium and Schizochytrium using non-chloride salts to produce a micro-floral biomass having omega-3 highly unsaturated fatty acids, U.S. Pat. No. 5,340,742 A).

The use of lipid-containing biomass of marine organisms as a carrier for active substances has not been described so far.

In the production of microparticles and nanoparticles there already exist several known production methods on the basis of lipids or polymers. What has already been described e.g. are methods for the production of lipid-microparticles on the basis of phospholipids, which microparticles have antimycotic properties and can be employed in the field of pharmaceutics and cosmetics (DE 69 00 2905 T2). Other methods describe lipid-nanoparticles on the basis of extracted mono-, di- and triglycerides, oils or waxes. By means of these obtained lipids one can also encapsulate pharmaceutically active substances (WO 94/20072 A1). Further lipid nanoparticles also describe lipid-based substances for parenteral application (WO 98/56362 A1). Specific techniques for the production of lipid-nanoparticles are also presented (e.g. EP 0 526 666 A). Until now however, no lipid-containing microparticles and nanoparticles on the basis of marine organisms (microalgae and macroalgae, thraustochytrids, marine bacteria) and their production have been described in literature.

Staphylococcus aureus is a ubiquitous commensal. About 30% of the world population represents permanent and asymptomatic hosts of S. aureus. This common and asymptomatic colonization leads to an often unrecognized spread.

On the other hand, S. aureus also is a common cause of serious infections and of sepsis, in particular in immunosuppressed persons.

An alarming development is the rapid formation of multiresistant S. aureus strains (MRSA). For reason of this multiresistance, infections with MRSA are difficult to control by therapy.

The occurrence of the MRSA-strains however is yet substantially restricted to hospitals until now. For this reason, hospitalization—irrespective of the primary disease—poses a large risk. About 10% of the received patients come down with nosocomial infections. One estimates a frequency of 600,000 to 800,000 cases of infections with multiresistant pathogens being acquired in hospitals (Hyg. Med. 26 (2001)183). Staphylococci are the most important pathogens, in case of which one can expect a complete failure of antibiotic therapy. A skin care counteracting the colonization with multiresistant pathogens can reduce the risk of nosocomial infections in case of hospitalization. If it was possible to prevent only a part of these infections, this would be a great success.

In order to decolonize occurring MRSA-populations it is nowadays common practice to use bactericidal substances. This however also kills the native population at the respective locus; therefore, in case of a novel colonization of the skin, the colonization with MRSA is even supported.

Recently, it has been published, that antimicrobial peptides can protect the skin from an invasive bacterial infection (V. Nizet, T. Ohtake, X. Lauth, J. Trowbridge, J. Rusdill, R. A. Dorschner, V. Pestonjamasp, J. Piraino, K. Huttner, R. L. Gallo: Innate antimicrobial peptide protects the skin from invasive bacterial infection. Nature 414, 454-457 (2001)). These peptides are produced on the surface of epithelial cells and in neutrophils and realize a very early defence against intruded pathogens. They cause cellular effects being clearly distinct from their antibacterial activity in vitro. The first critical step in a causal link finally resulting in infection, is the realisation of adhesion of the pathogen to appropriate receptors on the skin surface. This adhesion in many cases is introduced by the binding of proteins arranged on the pathogen's surface to carbohydrate-receptors of the host; besides this, also protein-protein-interactions and the binding of polysaccharides of the pathogen to skin receptors also play a role in this process (Relman D, Tuomanen E, Falkow S et al., Cell 1990; 61: 1375-1382; Kanbe T., Cutler J E. Infect Immun 1994; 62: 1662-1668).

It is thus the object of the present invention to provide novel active substances and carriers for active substances, the features of which constitute an improvement in comparison to the state of the art and which can be used for various purposes.

The object is achieved by pharmaceutically or cosmetically active agents obtained from marine organisms (microalgae and macroalgae, thraustochytrids, marine bacteria), which are produced by converting these organisms' biomasses into micro- and nanoparticles. According to the invention, biomasses of marine organisms (microalgae, macroalgae, thraustochytrids, marine bacteria) were converted in an economically advantageous, direct way into the novel substances having specific properties. We surprisingly found to have realized a particularly efficient utilization of the health-supporting components of the lipid-containing marine organisms and to have enabled various applications, which cannot be reached with the native biomasses.

The method according to the invention leads to novel, valuable products, which cannot be obtained by the known ways, this method comprising three alternatives:
1. Homogenisation Method

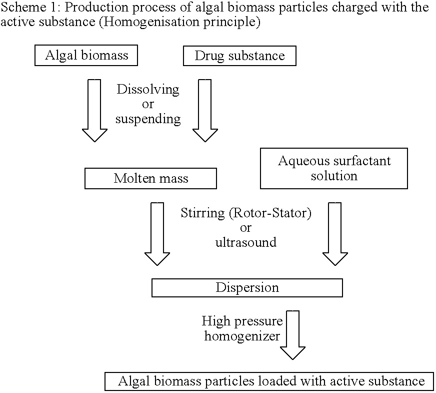

The lipid-containing marine microorganisms first are heated, so as to liquefy the fatty acids contained therein. One or more active substances (solid or liquid) are added to this biomass (scheme 1). The active substance is suspended, dispersed or adsorbed in the fatty acids of the cyanobacteria or, respectively, of the total lipid-containing marine microorganisms. In parallel, a surfactant-water mixture is prepared. This surfactant-water mixture is heated to a temperature above the fatty acids' melting points. The two phases are then combined at the selected temperature. In the following, a pre-suspension is produced by means of a stirring machine (rotor-stator principle) or by means of ultrasound. The pre-suspension is then homogenized by means of high pressure homogenizer, wherein the number of homogenizing cycles and the working pressure is selected according to the desired particle size and stability of the preparation. Between the individual cycles it has to be secured, that the production temperature has to be adjusted again and again. The surfactant serves to stabilize the suspension.

If the production process poses problems in respect to the temperature (e.g. heat-sensitive active substances), there exists the possibility to realize the entire process also at room temperature. In this case, the method is performed in a similar way as described above, wherein the active substance is adsorbed to the lipid-containing marine microorganisms or is dispersed in the presence of a little amount of added water.
2. Solvent-Homogenisation Method

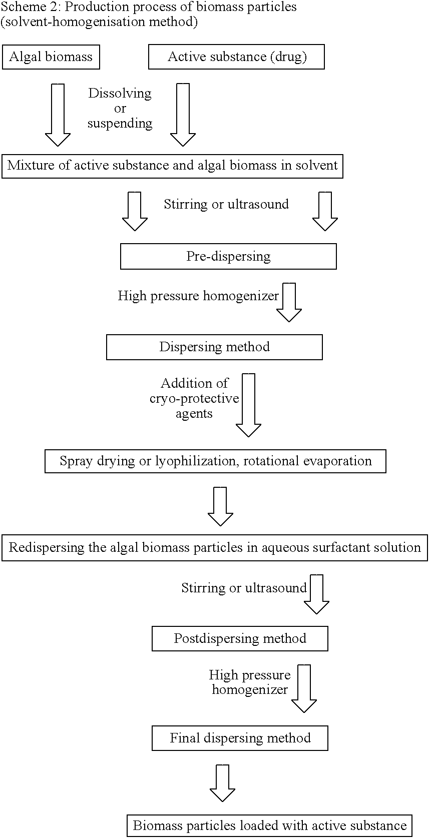

The lipid-containing marine microorganisms and the active substance are suspended in a vaporizable organic solvent. Thereafter, this mixture is pre-dispersed (stator-rotor principle or ultrasound), homogenized (high pressure homogenizer) and spray dried or lyophilized in the following (scheme 2). In case of using lyophilization, it has to be respected that one must add appropriate cryo-protective agents. Moreover, there also exists the possibility to remove the organic solvent by means of suitable evaporation devices (e.g. rotary evaporators). Then, the particles obtained from the lipid-containing marine microorganisms can be redispersed in appropriate aqueous surfactant solutions. Thereafter, a further dispersing process (stator-rotor principle or ultrasound) and homogenisation (high pressure homogenizer) are required.
3. Solvent-Emulsion Method

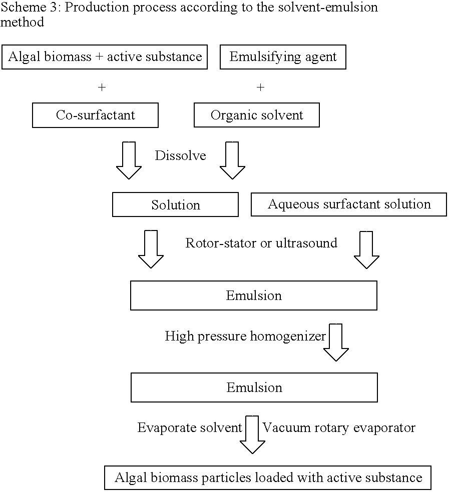

This method is based on the preparation of an emulsion of water and of a solution of the algal biomass active substance in a suitable organic solvent (scheme 3). To this aim, an emulsifying agent for dispersing the algal biomass active substance is employed.

The emulsifying agent and the algal biomass are dissolved in a suitable organic solvent. An aqueous phase containing a water-soluble co-surfactant is added to this solution. Then, this mixture is pre-dispersed (stator-rotor principle or ultrasound). After a homogenisation step by means of a high pressure homogenizer, the organic solvent is removed by evaporation, wherein the biomass containing the active substance recrystallizes in the form of solid particles.

The employment of these methods leads to novel marine biomass/active substance-particles with a mean diameter between 10 nm and 10 μm depending on the preparation method.

The agents according to the invention are effective also in the absence of an additionally added active substance, since the method according to the invention improves the availability of the components of the lipid-containing marine organisms.

Thus, it has already been surprising, that non-bactericidal natural substances being components of marine organisms, such as norlichexanthone, by means of the inventive method gain the potential to inhibit MRSA-growth in vitro.

It has moreover been surprising, that non-bactericidal natural active substances like ubiquinone derivatives commonly occurring in nature or hydroxylated aromatic compounds, as they have been discovered in various marine fungal species, by means of the inventive method as well inhibit the growth of MRSA in vitro. The use of these substances, which are known as such, for the defeat of MRSA, has not yet been described. Moreover, it is known, that the mentioned natural substances do not kill staphylococci on the skin to a sufficient degree. Thus, one has achieved a synergistic effect by the method according to the invention.

In transfer experiments from skin being contaminated with MRSA (donor) to skin being low in germs (acceptor) it has surprisingly become obvious, that a transfer of MRSA can be largely prevented, if the skin was treated with a synergistically acting combination of lipids (as components of marine organisms), immunostimulating agents, radical scavengers and xanthones of the general formula before the transfer of pathogens took place. Evidently, this synergistic combination already disturbs very early stages of the causal link in the transfer of pathogenic microorganisms to the actual infection, so that already the process of colony formation can be prevented.

Specific properties are achieved, if aggregates of the lipid-containing marine organisms and clay minerals (phyto-silicates) are contained in the micro- and nanoparticles produced according to claim 1. Specific characteristics of these micro- and nanoparticles are their large surface, their high ionic exchange capacity, their high swelling capacity and their capability to store extremely variable active substances within the layers of the silicate structure. Surprisingly, it was found that the aggregates produced in this manner significantly support cell growth.

Particularly advantageous is the use of clays being similar to bentonite in respect to their crystal structure. Bentonite not only exhibits a very high proportion of particles with a size of <2 μm, but also displays a high ion exchange capacity of 0.76 meq/g, an extremely large specific surface of 562 m²/g and the strongest influence on the growth of amnion epithelium cells.

A particularly advantageous embodiment of the invention is the use of lipid-containing marine organisms, which have e.g. been cultivated in the presence of clay minerals. For reason of their large specific surface, the micro- and nanoparticles produced on this basis are excellently suited as carriers for active substances, especially because the release of the active substance can be controlled very well due to the portion and composition of the mineral component.

The principles presented are suitable to store various caring components and/or mineral substances and/or radical scavengers and/or vitamins and/or dietary supplements within the biomasses. Thereby, cosmetics are enabled to utilize valuable algal components like proteins, mineral substances and vitamins, and polyunsaturated fatty acids like γ-linolenic acid, arachidonic acid, eicosapentaenoic acid and docosahexaenoic acid in a particularly advantageous form.

Object of the invention thus also is a composition of biomasses of marine organisms and mineral substances and/or radical scavengers and/or dietary supplements and/or vitamins, in particular vitamin C.

Additionally, according to the invention, one may incorporate one or more pharmaceutically or cosmetically active substances into the micro- and nanoparticles.

The addition of xanthones (e.g. norlichexanthone) or their derivatives and/or ubiquinones with a chain length from n=1 to n=15 and/or inorganic thiocyanates and/or hydrothiocyanates of organic bases and/or trihydroxybenzaldehyde or its derivatives have proven to be advantageous.

According to the invention, biomasses consisting of marine organisms can be used in combination with thiocyanates and/or hydrothiocyanates of organic bases and/or trihydroxybenzaldehyde or its derivatives also without converting said biomasses into micro- and nanoparticles and can be employed as cosmetic or pharmaceutical agents or can be used for the production of pharmaceutical and cosmetic agents.

The production according to the invention has the advantage, that the release of the incorporated substances can be controlled by the selection of the temperature, the active substances, the proportion of clay minerals and of the surfactants. According to the invention, it is advantageous to disperse the particles in distilled water or in an aqueous medium containing additives like electrolytes, polyones, mono-, di- and polysaccharides, agents for isotonic regulation, buffer substances, antifreeze agents and preserving agents. In order to achieve the inventive purpose, the addition of one or more dispersion-stabilizing substances may be necessary. An advantageous embodiment is characterized in that the biomass may be supplemented with one or several active substances, vitamins or dietary supplements in a solid and/or liquid form. It is appropriate to suspend, disperse or adsorb the added active substances in the fatty acids of the biomass. According to the invention, the biomass is united with a surfactant-water mixture in a further production step. It is advantageous to first produce a pre-suspension by means of a stirring machine (rotor-stator principle) or by means of ultrasound. According to the invention, this pre-suspension is then homogenized by means of a high pressure homogenizer, wherein the number of homogenizing cycles and the working pressure are selected in dependence on the particle size and stability of the formulation desired for the respective purpose.

In another, also inventive production method, the biomass and the active substances are suspended in a vaporizable organic solvent. Thereafter, this mixture is pre-dispersed (stator-rotator principle or ultrasound) and homogenized (high pressure homogenizer). In the following, the solvent is removed by spray drying or lyophilization or by means of rotational evaporation. If necessary, the biomass can be redispersed in appropriate aqueous surfactant solutions, then be post-dispersed (stator-rotator principle or ultrasound) and finally be homogenized (high pressure homogenizer). If necessary, a co-emulsifying agent or an emulsifying agent for dispersing the biomass-active substance mixture can be employed.

The microparticles and nanoparticles produced according to the described method allow for a use in very diverse fields. Surprisingly, there was a significantly improved bioavailability of the novel substances in comparison to the pure substances. This allows for a use of the particles as caring components in cosmetic products alone or in combination with other care products. The novel substances can be readily be incorporated into other care complex bases. The formulations according to the invention have a particular softening and caring effect on the skin. Just as fat emulsions, the particles display only a low systemic toxicity and hardly any cytotoxicity.

Absolutely surprisingly, it was found, that extracts obtained from the biomass show antibacterial effects and in vitro even clearly reduced the growth of the multiresistant staphylococci. After their conversion into micro- and nanoparticles, the components in the marine organisms support the skin's natural barrier function. The binding of a pathogenic microorganism to a host receptor is the critical early step in the development of a colonization or infection. Already this early step can surprisingly be influenced by the formulations according to the invention.

The present invention for the first time allows for the use of lipid-containing marine organisms as carriers for active substances like e.g. antibiotics.

The invention enables the use of biomasses of lipid-containing marine organisms in the form of micro- and nanoparticles as pharmaceutically or cosmetically active agents and as dietary supplements. It is moreover possible to use the lipid-containing marine organisms in the form of micro- and nanoparticles for the production of cosmetics or medicines or foodstuffs, and also for dietary products. A combination with other cosmetic agents or drugs is also practicable.

The invention can be used to prevent the binding of nosocomially important air-spread germs to skin or tissue receptors and/or their growth on said skin and tissues. The combination of biomasses of marine organisms with vitamins, in particular with vitamin C, has proven to be especially effective.

The use according to the invention also encompasses the prophylaxis of nosocomial infections and is particularly suitable for inhibiting staphylococci, in particular MRSA (multiresistant Staphylococcus aureus strains) to clean up a skin contaminated with MRSA.

A particularly effective skin care product was obtained by adding as active substances xanthone derivatives with the general formula

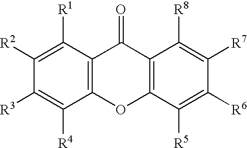

wherein R1-R8 can be selected from the substituents listed in table 1.

TABLE 1
H, OH, OMe, OAc

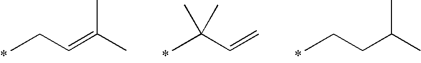

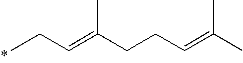

Me, Ac, CH₂OH, CHO, CF₃, COOH, COOMe, CN, CONH₂
Cl, F, NO₂, NH₂, NHAc, NMe₂

Additionally contained as radical scavengers may be tocopherols and/or benzoquinones with the general formula 2, with a chain length of n=1 to n=15, with the residues R1-R3, wherein R1, R2 and R3 represent hydrogen, halogen, alkyl or alkoxy group, with R4 as an isoprenoid side chain with 1 to 10 isoprenoid elements X or, as an aliphatic side chain, with 1 to 10 CH₃-residues (formula 3).

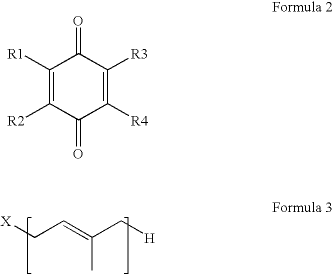

The agents according to the invention may contain dihydroxybenzaldehyde or its derivatives having the general formula 4, containing the residues R1, R2, R3, R4 and R5 as hydrogen, halogen, alkyl or alkane groups.

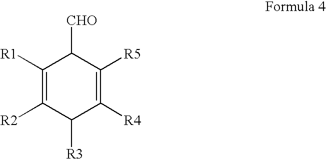

Many lipid-containing marine organisms contain substances, which have the potential to act as unspecific immunostimulating agents, i.e. they stimulate leucocytes and act as activators of the reticuloendothelial system. When the biomass of these organisms is converted into micro- and nanoparticles according to the invention, said substances allow for further applications. Advantageous e.g. is the employment in cover materials for wound treatment. Micro- and nanoparticles doped with antibiotics according to the invention allow for a controlled release of the antimicrobial active substances and a simultaneous immunostimulation. Another advantageous option is to additionally dope these micro- and nanoparticles with inorganic thiocyanates or hydrothiocyanates of organic bases.

According to the invention, the micro- and nanoparticles may additionally contain DNA, thus allowing for their use for a gene transfer.

Besides the use in cosmetic products, also the application in the dietary field and in the field of foodstuff technology are part of the invention. The lipophilic marine organisms' great wealth in valuable components—after a conversion into micro- and nanoparticles according to the invention—also allows for a goal-directed substitution in case of deficiency syndromes. Lipids, vitamins, mineral substances and other valuable components of the marine organisms can be used more effectively by the mammalian organism after the marine organisms have been converted into micro- and nanoparticles. Enrichment with dietary supplemental substances can be readily performed when using the methods according to the invention. The following groups of marine organisms comprise components, which are exclusively found in them in a suitable composition and concentration and which by means of the inventive conversion into micro- and nanoparticles can be made available for use:

Cyanobacteria from the class Oscillatoriales, in particular the strains SPH 03, SPH 04, SPH 05, SPH 06, SPH 09, SPH 10, SPH 11, SPH 12, SPH 13, SPH 14, SPH 20, SPH 21, SPH 22, SPH 23, SPH 25, SPH 26, SPH 29, SPH 32, SPH 34, SPH 37.

Cyanobacteria from the class Nostocales, in particular the strains SPH 18, SPH 20, SPH 27, SPH 28, SPH 38

Cyanobacteria from the class Chroococcales with particular regard to the strains SPH 07a, SPH 07b, SPH 08, SPH 14, SPH 16, SPH 17, SPH 24, SPH 33, SPH 36, SPH 39, SPH 40, SPH 43 and the class Stigonematales

Macroalgae from the genera Asparagopsis, Cystoseira, Codium, Dictyota, Dictyopteris, Enteromorpha, Fucus, Gelidium, Gracilaria, Gracilariopsis, Halopteris, Hypoglossum, Laurencia, Plocamium, Polyneura, Sargassum, Solieria, Ulva

Thraustochytrids from the genera Schizochytrium and Thraustochytrium

Marine bacteria from the genera Photobacterium, Shewanella and Colwellia.

It is particularly advantageous to have the option produce the particles by means of stirring machines (rotor-stator principle) and high pressure homogenizers, which have been used for decades in the production of fat emulsions for parenteral nutrition and thus are available for an industrial scale production of biomass particles. They are accepted by the national administrative bodies for the production of parenteralia and thus do not require new complicate admission procedures.

The conversion of biomasses of lipid-containing marine organisms according to the invention moreover provides further applications. The lipids bind to plastic surfaces. This allows for the employment of the inventively doped micro- and nanoparticles obtained from the lipid-containing marine organisms in slow-release systems for the prevention of infections associated with implants, preferably of infections associated with catheters. The stable biomatrix allows for the protection of the incorporated active substance against chemical degradation and secures a retarded release of the pharmacon. By the selection of appropriate surfactants and active substances however, it is also possible to achieve an accelerated release of the active substances from the particle surface, when said active substances are located at the surface, thus providing an inventive, controlled release of active substances. The method according to the invention allows to sterilize the materials doped with the inventive micro- and nanoparticles.

In contrast to polymer nanoparticles, the novel particles obtained from marine biomass are biodegradable.

The characteristics of the invention are fully defined by the elements of the claims and by the description, wherein both single characteristics and more characteristics in the form of combinations constitute advantageous embodiments, for which protection is applied for by this specification.

The basic idea of the invention is the combination of known elements (biomasses of lipid-containing marine organisms) and new elements (the conversion of said biomasses into micro- or nanoparticles), these elements having mutual influence on each other and in their new total effect providing an application advantage and the desired positive results, i.e. the first provision of active substance carriers on the basis of lipid-containing marine microorganisms, the realization of bioavailability of the components/compounds of these organisms, the lending of novel properties to substances so far lacking pharmaceutical activity, and the possibility to control the release of the incorporated substances by the choice of temperature, active substances, clay mineral portion and of the surfactants.

The invention will be described in the following by means of examples without being limited to them.

### EXAMPLES

### Example 1

### Production of Micro- and Nanoparticles from Biomass of Cyanobacteria from the Order Chroococcales

We used cyanobacteria from the strain B 30, which was obtained as an own isolate from the “Jasmunder Bodden” of the Baltic Sea. Extracts of different polarity from this strain did not show any antimicrobial activity in screening tests.

TABLE 2
Formula of the biomass B30 - vitamin C - micro- and nanoparticles

| Substance | Amount in g |
|---|---|
| Biomass | 5.00 |
| Emulsifying agent (Plantacare 2000) | 0.05 |
| Demineralised Water | 45.00 |
| Cycles of homogenization | 4 |

The biomass is heated to a temperature of 50° C. In a separate approach, an aqueous solution of the emulsifying agent is heated to the respective temperature (50° C.). Thereafter, both phases are unified at the desired homogenisation temperature. The mixture is then processed by means of an Ultra Turrax T25 (Fa. Janke und Kunkel GmbH & Co KG (Staufen, Germany)) in an emulsifying process at 8000 rotations per minute for a period of 30 seconds. Thereafter, the suspension is homogenized for four times at a pressure of 500 bar and a temperature of 50° C. by means of a piston gap high pressure homogenizer, Micron Lab 40 (APV-Gaulin, Lübeck).

### Example 2

### Production of Vitamin C-Containing Micro- and Nanoparticles from Biomass of Cyanobacteria from the Order Chroococcales

We used cyanobacteria from the strain B 30, which was obtained as an own isolate from the “Jasmunder Bodden” of the Baltic Sea. Extracts of different polarity from this strain did not show any antimicrobial activity in screening tests.

TABLE 3
Formula of the biomass B30 - vitamin C - micro- and nanoparticles

| Substance | Amount in g |
|---|---|
| Biomass | 5.00 |
| Emulsifying agent (Plantacare 2000) | 0.05 |
| Demineralised Water | 45.00 |
| Vitamin C | 5 |
| Cycles of homogenisation | 4 |

In the following, 5 g of vitamin C were integrated into the biomass. In a separate approach, an aqueous solution of the emulsifying agent is heated to the respective temperature (50° C.). Thereafter, both phases are unified at the desired homogenisation temperature. The mixture is then processed by means of an Ultra Turrax T25 (Fa. Janke und Kunkel GmbH & Co KG (Staufen, Germany)) in an emulsifying process at 8000 rotations per minute for a period of 30 seconds. Thereafter, the suspension is homogenized for four times at a pressure of 500 bar and a temperature of 50° C. by means of a piston gap high pressure homogenizer, Micron Lab 40 (APV-Gaulin, Lübeck).

The two micro- and nanoparticles B30 and vitamin C were mixed with each other.

### Example 3

### Production of Micro- and Nanoparticles from Biomass of Cyanobacteria from the Genus Spirulina

We used commercially available cyanobacteria from the genus Spirulina.

TABLE 4
Formula of the Spirulina-micro- and nanoparticles

| Substance | Amount in g |
|---|---|
| Biomass | 5.00 |
| Emulsifying agent (Plantacare 2000) | 0.05 |
| Demineralised Water | 45.00 |
| Cycles of homogenisation | 4 |

The biomass is dispersed at a temperature of 25° C. in an aqueous solution of the emulsifying agent. The mixture is then processed by means of an Ultra Turrax T25 (Fa. Janke und Kunkel GmbH & Co KG (Staufen, Germany)) in an emulsifying process at 8000 rotations per minute for a period of 30 seconds. Thereafter, the suspension is homogenized for four times at a pressure of 500 bar and a temperature of 50° C. by means of a piston gap high pressure homogenizer, Micron Lab 40 (APV-Gaulin, Lübeck).

### Example 4

### Production of Micro- and Nanoparticles from Biomass of Cyanobacteria from the Genus Oscillatoria

We used cyanobacteria from the species Oscillatoria redekei HUB 051.

TABLE 5
Formula of the Oscillatoria redekei HUB 051-particles

| Substance | Amount in g |
|---|---|
| Biomass | 5.00 |
| Emulsifying agent (Plantacare 2000) | 0.05 |
| Demineralised Water | 45.00 |
| Cycles of homogenisation | 4 |

The biomass is dispersed at a temperature of 25° C. in an aqueous solution of the emulsifying agent. The mixture is then processed by means of an Ultra Turrax T25 (Fa. Janke und Kunkel GmbH & Co KG (Staufen, Germany)) in an emulsifying process at 8000 rotations per minute for a period of 30 seconds. Thereafter, the suspension is homogenized for four times at a pressure of 500 bar and a temperature of 50° C. by means of a piston gap high pressure homogenizer, Micron Lab 40 (APV-Gaulin, Lübeck).

### Example 5

### Production of Micro- and Nanoparticles from Biomass of Cyanobacteria from the order Nostocales

We used cyanobacteria from the order Nostocales.

TABLE 6
Formula of the Nostocales micro- and nanoparticles.

| Substance | Amount in g |
|---|---|
| Biomass | 5.00 |
| Emulsifying agent (Plantacare 2000) | 0.05 |
| Demineralised Water | 45.00 |
| Cycles of homogenisation | 4 |

The biomass is dispersed at a temperature of 25° C. in an aqueous solution of the emulsifying agent. The mixture is then processed by means of an Ultra Turrax T25 (Fa. Janke und Kunkel GmbH & Co KG (Staufen, Germany)) in an emulsifying process at 8000 rotations per minute for a period of 30 seconds. Thereafter, the suspension is homogenized for four times at a pressure of 500 bar and a temperature of 50° C. by means of a piston gap high pressure homogenizer, Micron Lab 40 (APV-Gaulin, Lübeck).

### Example 6

### Production of Micro- and Nanoparticles from Biomass of Cyanobacteria from the Order Chroococcales with Vitamin C According to the Solvent Method

As biomass, we used the strain B 30 described in example 1.

TABLE 7
(solvent method)

| | | |
|---|---|---|
| Water | 45.00 | g purified, filtrated water |
| Organic solvent | 25 | ml n-hexane |
| Biomass | 0.5 | g Chroococcales cyanobacteria |
| Vitamin | 5 | g vitamin C |
| Surfactant | 0.5 | g Plantacare |

First, the biomass is dissolved in n-hexane, thereby disentangling the lipids from the biomass. The biomass is stirred in the solvent for about 3 hours. Thereafter, the solvent is removed by means of a rotary evaporator. Thereby, a lipid layer is formed at the surface of the flask. For redispersing, a vitamin C-containing solution is required: vitamin C is added to a Plantacare solution and, after a dispersing process of 90 seconds, the mixture is homogenized for four times. The resulting, vitamin C-containing solution is introduced into the flask containing the lipid layer. The lipid layer is released during the subsequent rotation of the flask, thereby forming capsule-like micro- and nanoparticles. The rotation takes about 10 hours at a temperature of 40° C.

### Example 7

### Production of Micro- and Nanoparticles from Biomass Plus Norlichexanthone

TABLE 8
Formula of the biomass-norlichexanthone micro- and nanoparticles

| Substance | Amount in g |
|---|---|
| Active substance (norlichexanthone) | 0.05 |
| Biomass (cyanobacteria Spirulina) | 5.00 |
| Emulsifying agent (Plantacare 2000) | 0.05 |
| Demineralised water | 45.00 |
| Cycles of homogenisation | 4 |

The biomass is heated to a temperature of 50° C. and, thereafter, the employed marine active substance norlichexanthone is dispersed or dissolved therein. In a separate approach, an aqueous solution of the emulsifying agent is heated to the respective temperature (50° C.). Then, both phases are unified at the desired homogenisation temperature. The mixture is then processed by means of an Ultra Turrax T25 (Fa. Janke und Kunkel GmbH & Co KG (Staufen, Germany)) in an emulsifying process at 8000 rotations per minute for a period of 30 seconds. Thereafter, the suspension is homogenized for four times at a pressure of 500 bar and a temperature of 50° C. by means of a piston gap high pressure homogenizer, Micron Lab 40 (APV-Gaulin, Lübeck).

### Example 8

### Production of Micro- and Nanoparticles from Biomass Plus Vitamin E

TABLE 9
Formula of the biomass-vitamin E micro- and nanoparticles

| Substance | Amount in g |
|---|---|
| Active substance (vitamin E) | 0.05 |
| Biomass (cyanobacteria Spirulina) | 5.00 |
| Emulsifying agent (Plantacare 2000) | 0.05 |
| Demineralised water | 45.00 |
| Cycles of homogenisation | 4 |

The vitamin E is dispersed in the biomass. In a separate approach, an aqueous solution of the emulsifying agent is produced. The mixture is then processed by means of an Ultra Turrax T25 (Fa. Janke und Kunkel GmbH & Co KG (Staufen, Germany)) in an emulsifying process at 8000 rotations per minute for a period of 30 seconds. Thereafter, the suspension is homogenized for four times at a pressure of 500 bar and a temperature of 50° C. by means of a piston gap high pressure homogenizer, Micron Lab 40 (APV-Gaulin, Lübeck).

### Example 9

### Production of Micro- and Nanoparticles from Biomass Plus Provitamin Q10

TABLE 10
Formula of the biomass-provitamin Q10 micro- and nanoparticles

| Substance | Amount in g |
|---|---|
| Active substance (provitamin Q10) | 0.05 |
| Biomass (cyanobacteria Spirulina) | 5.00 |
| Emulsifying agent (Plantacare 2000) | 0.05 |
| Demineralised water | 45.00 |
| Cycles of homogenisation | 4 |

Provitamin Q10 is dispersed in the biomass. In a separate approach, an aqueous solution of the emulsifying agent is produced. The mixture is then processed by means of an Ultra Turrax T25 (Fa. Janke und Kunkel GmbH & Co KG (Staufen, Germany)) in an emulsifying process at 8000 rotations per minute for a period of 30 seconds. Thereafter, the suspension is homogenized for four times at a pressure of 500 bar and a temperature of 50° C. by means of a piston gap high pressure homogenizer, Micron Lab 40 (APV-Gaulin, Lübeck).

### Example 10

### Test of the Micro- and Nanoparticles “Biomass B30/Vitamin C” Produced According to the Examples 1 and 2, in the Animal Model (Cow Udder Teats)

The two substances were mixed in a ratio of 1:1.

Methodical Approach:

The udder teats were processed one hour after the cow had been killed. The fat layer was removed from the teats. Thereafter, the teats were placed on metallic rods of appropriate size and fixed thereto by means of clamps. The teats were cleaned with 70% alcohol. 20 μl of the test substance were pipetted onto the teats and spread by rubbing by means of a glass spatula, followed by drying at room temperature for 30-45 minutes. The contamination was performed by applying 10 μl of the Northern German strain MF 0.5, diluted 1:10. After an incubation at 30° C. for 1.5 hours, the skin sections were smoothed down on Müller-Hinton plates, followed by an incubation at 37° C.

Evaluation:

In all controls we found germ numbers>100. These results were used to calculate the mean germ number n and the statistical distribution.

In the analyses of the preparations we found germ numbers between 0 and 100. These results were used to calculate the mean germ number m on the basis of the Poisson-distribution according to the following equation: m=1n (number of samples without detected germs/total number of samples).

Statistical Evaluation:

The number of skin areas with and without positive germ detection for allowing the evaluation without treatment and after treatment with the test product, was analysed in respect to statistical significance by means of the Chi Square Test.

Results:

The testing of micro- and nanoparticles produced according to example 1 in the skin model (cow udder teat) led to a pronounced reduction of the transferred contamination with MRSA. The number of reiterations at the cow udder teat was 163 for the untreated controls. S. aureus was detectable in all of these 163 samples. The mean germ number was 759. S. aureus was also detected in all of the samples treated with wool wax alcohol ointment. After the treatment with the test products, 11 skin areas showed positive germ detection and 11 areas showed no detectable germs. This results in mean germ number of 0.7 to be expected (FIG. 1). The germ number reduction is highly significant.

### Example 11

### Testing of Micro- and Nanoparticles “Biomass B30/Vitamin C” Produced According to Examples 1 and 2, in the Animal Model Mouse Ear

Methodical Approach:

As a test model, we used mouse ears. The donor animals as the source of infection remained untreated. The acceptor animals were treated with “biomass B30/vitamin C” produced according to example 1, this treatment being performed once a day for 3 days. To this aim, 10 μl of the test substance were pipetted onto the test system and spread by rubbing by means of a glass spatula, followed by drying at room temperature. The animals were killed after four days. The contamination of the donor animals was performed by applying 5 μl of the Northern German strain MF 0.5, diluted 1:10. For this, an untreated ear was contaminated and then incubated for 90 minutes at 30° C. The ears treated with biomass were mounted on appropriate cachets. The contaminated ears were pressed under the application of pressure onto the untreated ears for 10 seconds.

After incubation at 30° C. for 1.5 hours, the skin areas of the acceptor ears were smoothed down on Müller-Hinton plates and incubated at 37° C.

Evaluation:

The evaluation and the statistical analysis were performed according to example 9.

Results:

The testing of the micro- and nanoparticles produced according to examples 1 and 2 also in the donor-acceptor experiment, in which the transmission of infection via skin contact was simulated, showed a significant reduction of transmitted germs.

The number of reiterations at the mouse ear was 163 for the untreated controls, thereby positively detecting the growth of germs on the skin areas. In case of the pre-treatment with the preparations according to examples 1 and 2, 12 skin areas were with and 5 were without positive germ detection. In the end, only an average of 1.2 germs/cm² is to be expected after the respective pre-treatment (FIG. 2).

### Example 12

### Testing of the Micro- and Nanoparticles “Biomass B30/Vitamin C” Produced According to Examples 1 and 2, in the Potbelly Pig

Methodical Approach:

For the testing we used a potbelly pig. 2 hours after the animal had been killed, skin was excised from the bottom side of the belly in neighbourhood to the teats. The fat layer was largely removed and the skin cut into pieces. These pieces were fixed to mounting devices, so that approximately 1 cm² was available for treatment. The skin areas were shaved or the bristles were cut away with scissors, followed by cleaning the skin 2 times with 70% ethanol. 20 μl of test substance were applied and incubated for 45 minutes. The contamination was performed with 10 μl of the Northern German strain MF 0.5, diluted 1:10. After an incubation period of 1.5 hours at 30° C., the skin areas were smoothed down on Müller-Hinton plates and incubated at 37° C. The germs were counted after the incubation.

Result

In spite of the performed skin disinfection, many coagulase-negative apathogenic staphylococci of the normal skin flora of the pig were detectable in the smear test. The coagulase-positive Staphylococcus aureus strains, which had been used for the contamination, were numerously detected besides S. epidermidis in the untreated control, whereas in the animals treated according to the invention only coagulase-negative colonies were detected.

### Example 13

### Testing of Micro- and Nanoparticles “Biomass B30/Vitamin C” Produced According to Example 6, in the Skin Model According to Example 10

Result:

The number of reiterations at the cow udder teat was 158 for the untreated controls. After the application of the micro- and nanoparticles, the number of skin areas with positive germ detection was 2, whereas 4 skin areas were without positive germ detection. This means, that after treatment with the inventive preparation according to example 5, an average of only 0.4 germs/cm² is to be expected. The reduction of the germ numbers is highly significant in the Chi Square Test.

### Example 14

### Testing of Micro- and Nanoparticles “Biomass B30/Vitamin C” Produced According to Example 6, in the Skin Model According to Example 11

Results:

The testing of the micro- and nanoparticles produced according to example 6, in the donor-acceptor experiment being designed to simulate the transmission of infection via skin contact, showed a significant reduction of the transferred germs. There was 1 skin area with positive germ detection, but 5 skin areas without germ detection. This means, that after the inventive pre-treatment an average of only 0.18 germs was observed. The reduction of the transferred germs is highly significant in the Chi Square Test.

### Example 15

### Examination of the Micro- and Nanoparticles Produced According to the Examples in the Skin Model According to Example 10

Results:

The micro- and nanoparticles norlichexanthone-vitamin E-Q 10 (solvent method) were produced according to the examples 7, 8, 9 and mixed in a ratio of 1:1:1. The testing was performed according to the method presented in example 10.

Result:

The number of tested preparations with norlichexanthone was 22. The number of skin areas with positive germ detection was 19 and the number of skin areas without positive detection was 3. Thus, an average of 2 germs is still to be expected after the pre-treatment with the inventive preparations according to example 8.

### Example 16

### Production of Ubiquinone Q1 Biomass Particles

TABLE 11
Formula of the ubiquinone-algal biomass particles

| Substance | Amount in g |
|---|---|
| Active substance (ubiquinone Q1) | 0.05 |
| Biomass (Oscillatoria redekei HUB051) | 5.00 |
| Emulsifying agent (Plantacare 2000) | 0.05 |
| Demineralised water | 45.00 |
| Cycles of homogenisation | 4 |

The biomass is heated to a temperature of 50° C., followed by dispersing or dissolving ubiquinone Q1 in the biomass. In a separate approach, an aqueous solution of the emulsifying agent is heated to the respective temperature (50° C.). Thereafter, the two phases are united at the desired homogenisation temperature. The mixture is then processed by means of an Ultra Turrax T25 (Fa. Janke und Kunkel GmbH & Co KG (Staufen, Germany)) in an emulsifying process at 8000 rotations per minute for a period of 30 seconds.

Thereafter, the suspension is homogenized for four times at a pressure of 500 bar and a temperature of 50° C. by means of a piston gap high pressure homogenizer, Micron Lab 40 (APV-Gaulin, Lübeck).

### Example 17

### Prevention of the Transmission of MRSA in Skin Contacts by the Preparation According to Example 15

Methodical Approach:

For these experiments we used mouse tails from mice maintained under sterile conditions. In order to exclude any foreign source of contamination, the tails were placed into 70% alcohol for 5 minutes before the beginning of the experiments and were then dried under the laminar box.

The donor mouse tails as the source of infection were contaminated by placing them into a diluted MRSA-culture (Northern German strain, MF-standard 0.5 or 0.3) for 30 s up to 4 min, followed by an incubation period of 24 h at 37° C. In smear tests using these donors, germ numbers of >100,000 were detected.

The test substances were rubbed in twice a day into the mouse tails, which simulate the recipient organism (acceptor).

The infective transmission from donor to acceptor was accomplished via skin contact with the donors for 30 s to 1 min on the shaker at 600 U/min. 2 h or 24 h after the contamination, the acceptors were smoothed down on blood-Müller-Hinton agar plates. After an incubation period of 24 h at 37° C., the colonies on the agar plates were counted.

Results:

TABLE 12
Germ numbers on mouse tails (acceptors) in dependence on the
pre-treatment after contact with MRSA colonized
mouse tails (donors).

| Donor | | | | | |
|---|---|---|---|---|---|
| | Contami- | Skin | Pre-treatment | Germ number | |
| McFarland- | nation | contact | of the | of the acceptor | |
| Standard | (min) | (min) | acceptor | 2 h | 24 h |
| | | 0.5 | | >10000 | >10000 |
| 0.5 | 5 | 1 | without | >10000 | >10000 |
| | | 0, 5 | | 143 | 2 |
| 0.5 | 5 | 1 | 2 d | 80 | 36 |
| | | | preparation | | |
| | | | according to | | |
| | | | example 15 | | |
| 0.5 | 3 | 0.5 | without | >10000 | >10000 |
| | | | 2 d | | 6 |
| | | | Preparation | | |
| | | | according to | | |
| | | | example 15 | | |
| 0.3 | 1 | 1 | without | | >10000 |
| | | | 2 d | | 2 |
| | | | Preparation | | |
| | | | according to | | |
| | | | example 15 | | |

When smoothing down the acceptors immediately after the skin contact, the plates are completely colonized (germ number>10,000). This equally applies to pre-treated acceptors and to the controls without pre-treatment. This means, that a massive transmission of MRSA can be well simulated by this model. In the experimental group and in the control group, the same germ numbers are transferred.

After 2 h or 24 h however, only low germ numbers can be detected in the pre-treated acceptors, whereas the germ numbers in the untreated controls are invariably high (Table 12). Thus, also in this experimental model one can demonstrate the interruption of the infective pathways by a pre-treatment with the formulations according to the invention.

### Example 18

### Determination of the Particle Size of the Micro- and Nanoparticles Produced According to Example 3

Methodical Approach:

The particle size was determined by photon correlation spectroscopy using a Malvernzizer III (Malvern, UK).

### Example 19

### Production of DNA-Containing Micro- and Nanoparticles from Biomass of Cyanobacteria from the Order Chroococcales

TABLE 13
Examples of formulas: Incorporation of DNA into the algal biomass

| Substance | Formula 1 | Formula 2 | Formula 3 |
|---|---|---|---|
| Active substance | 5% | 10% | 15% |
| herring sperm | | | |
| DNA) | | | |
| Base of algal | 5.00 g cyanobacteria | | |
| biomass of the | | | |
| order Chroococcales | | | |
| Emulsifying agent | 0.60 g | 1.25 g | 0.60 g |
| | Plantacare ® | Pluronic ®F-68 | Miranol |
| | | 68 | Ultra 32 |
| Demineralised | 45.00 g | | |
| Water | | | |
| Cycles of | 4 | 3 | 3 |
| homogenisation | | | |

When incorporating the DNA (Sigma-Aldrich, Deisenhofen (Germany)) in the algal biomass particles, the production parameters are slightly varied in order to avoid a destruction of the DNA:

Pre-homogenisation by means of the Ultra-Turrax: 30 sec

Homogenisation temperature: 55° C.

Homogenisation pressure: 500 bar

Number of homogenisation cycles: 2

In the first approach, the DNA in its dried form after the lyophilization is dispersed in the algal biomass and then homogenized. In the second approach, the DNA being dissolved in water is simply added to the aqueous solution of the emulsifying agent and dispensed together therewith in the heated algal biomass, as well followed by homogenisation.

### Example 20

### Production of Triamcinolone-Containing Micro- and Nanoparticles from Biomass of Cyanobacteria from the Order Chroococcales

TABLE 14
(solvent-emulsion method)

| | | |
|---|---|---|
| Water | 45.00 | g of purified, filtrated water |
| Organic solvent | 7.50 | g dichloromethane |
| Algal biomass (Spirulina) | 1.12 | g cyanobacteria (15% in relation to |
| | | dichloromethane) |
| Triamcinolone | 0.12 | g triamcinolone |
| Lipophilic | 0.38 | g Epikuron ® 170 (5% in relation to |
| emulsifying agent | | dichloromethane) |
| Co-surfactant | 0.11 | g Pluronic ® F-68 (1.5% in relation |
| | | to dichloromethane) |

The production of the triamcinolone-cyanobacteria microparticles is based on the production of an emulsion of water and a solution of the cyanobacteria containing the active substance triamcinolone in the solvent dichloromethane. After the homogenisation step, the organic solvent (dichloromethane) is removed by evaporation, wherein the algal biomass crystallizes in the form of solid nanoparticles. The emulsion or dispersion is stabilized by means of a suitable mixture of surfactants (Pluronic® F-68).

Used as machines were an Ultra-Turrax T25 (Firma Janke & Kunkel GmbH & Co KG (Staufen/Germany)), and a piston gap homogenizer, Micron Lab 40, from the company APV Gaulin (Lübeck, Deutschland) with 7 cycles and at 800 bar. Additionally, a rotary evaporator, Rotavapor R114, connected to a vacuum system, B178, from the Finna Büchi (Flawil/Switzerland), was employed at a pressure of 0.7 bar for 60 minutes.

### Example 21

### Production of Ubiquinone Q1-Algal Biomass Particles

TABLE 15
Formula of the ubiquinone-algal biomass particles

| Substance | Amount in g |
|---|---|
| Active substance (ubiquinone Q1) | 0.05 |
| Algal biomass | 5.00 |
| Emulsifying agent (Plantacare 2000) | 0.05 |
| Dernineralised water | 45.00 |
| Cycles of homogenisation | 4 |

The algal biomass is heated to a temperature of 50° C. and, thereafter, the employed active substance ubiquinone Q1 is dispersed or dissolved therein. In a separate approach, an aqueous solution of the emulsifying agent is heated to the respective temperature (50° C.). Thereafter, both phases are unified at the desired homogenisation temperature. The mixture is then processed by means of an Ultra Turrax T25 (Fa. Janke und Kunkel GmbH & Co KG (Staufen, Germany)) in an emulsifying process at 8000 rotations per minute for a period of 30 seconds. Thereafter, the suspension is homogenized for four times at a pressure of 500 bar and a temperature of 50° C. by means of a piston gap high pressure homogenizer, Micron Lab 40 (APV-Gaulin, Lübeck).

### Example 22

TABLE 16
Exemplary formulas for incorporating DNA in algal biomass

| Substance | Formula 1 | Formula 2 | Formula 3 |
|---|---|---|---|
| Active substance (DNA) | 5% | 10% | 15% |
| Algal biomass base | 5.00 g cyanobacteria | | |
| Emulsifying agent | 0.60 g | 1.25 g Pluronic ® | 0.60 g |
| | Plantacare ® | F-68 | Miranol |
| | | | Ultra 32 |
| Demineralised water | 45.00 g | | |
| Cycles of | 4 | 3 | 3 |
| homogenisation | | | |

When incorporating the DNA in the algal biomass particles, the production parameters are slightly varied in order to avoid a destruction of the DNA:

Pre-homogenisation by means of the Ultra-Turrax: 30 sec

Homogenisation temperature: 55° C.

Homogenisation pressure: 500 bar

Number of homogenisation cycles: 2

In the first approach, the DNA in its dried form after the lyophilization is dispersed in the algal biomass and then homogenized. In the second approach, the DNA being dissolved in water is simply added to the aqueous solution of the emulsifying agent and dispensed together therewith in the heated algal biomass, as well followed by homogenisation.

### Example 23

### Production of Triamcinolone-Algal Biomass Microparticles

TABLE 17
(solvent-emulsion method)

| | | |
|---|---|---|
| Water | 45.00 | g of purified, filtrated water |
| Organic solvent | 7.50 | g dichloromethane |
| Algal biomass | 1.12 | g cyanobacteria (15% in relation to |
| | | dichloromethane) |
| Triamcinolone | 0.12 | g triamcinolone |
| Lipophilic | 0.38 | g Epikuron ® 170 (5% in relation to |
| emulsifying agent | | dichloromethane) |
| Co-surfactant | 0.11 | g Pluronic ® F-68 (1.5% in relation |
| | | to dichloromethane) |

The production of the triamcinolone-cyanobacteria microparticles is based on the production of an emulsion of water and a solution of the cyanobacteria containing the active substance triamcinolone in the solvent dichloromethane. After a homogenisation step, the organic solvent (dichloromethane) is removed by evaporation, wherein the algal biomass crystallizes in the form of solid nanoparticles. The emulsion or dispersion is stabilized by means of a suitable mixture of surfactants (Pluronic® F-68).

Used as machines were an Ultra-Turrax T25 (Firma Janke & Kunkel GmbH & Co KG (Staufen/Germany)), and a piston gap homogenizer, Micron Lab 40, from the company APV Gaulin (Lübeck Deutschland) with 7 cycles and at 800 bar. Additionally, a rotary evaporator, Rotavapor R114, connected to a vacuum system, B178, from the Finna Büchi (Flawil/Switzerland), was employed at a pressure of 0.7 bar for 60 minutes.

### Example 24

### Influence of Micro- and Nanoparticles with Aggregates of Lipid-Containing Marine Organisms and Clay Minerals on the Growth of Amnion Epithelium Cells

Cyanophycea were cultivated up to 2 month with additives of bentonite, “Friedländer Ton” (a type of clay) and kaolin. Aggregates developed already after a short incubation period, whereas respective aggregates took 2 month to develop and developed only to a lesser extent in the controls containing no mineral additives. The mineral substance with the highest absorption capacity (bentonite) shows a lower tendency to form aggregates than kaolin with its small absorption capacity. The formation of aggregates and the absorption capacity of the aggregates formed can be controlled by the selection of the mineral components. The aggregates were subsequently processed by means of an Ultra Turrax T25 (Fa. Janke und Kunkel GmbH & Co KG (Staufen, Germany)) in an emulsifying process with 8000 rotations per minute for a period of 30 seconds.

When acting upon FL-cells, in particular the aggregates with bentonite significantly increased cell growth (p=<0.001). This influence is clearly stronger than the growth promoting effect of Friedländer Ton (p=0.024). In contrast thereto, the influence of kaolin is not significant (p=0.2786).

### Example 25

### Analysis of the Antibacterial Activity in the Agar Diffusion Inhibition Test

TABLE 18

| | | Trihydroxy- | Trihydroxy- |
|---|---|---|---|
| | | benzaldehyde, | benzaldehyde, |
| | Trihydroxy- | 3% in viscous | 10% in viscous |
| Pathogen | benzaldehyde | paraffin | paraffin |
| MRSA | 10 mm | 12 mm | 14 mm |
| “Norddeutscher” | | | |
| Epidemie- | | | |
| Stamm” | | | |

### Example 26

### Decontamination of MRSA-Populated Skin in the Mouse Tail Test

TABLE 19
Germ numbers on mouse tails (acceptors) in dependence on the
pre-treatment after the contact with MRSA-populated
mouse tails (donors).

| Donor | | | | | |
|---|---|---|---|---|---|
| | Contami- | Skin | Pre-treatment | Germ number | |
| McFarland- | nation | contact | of the | of the acceptor | |
| Standard | (min) | (min) | acceptor | 2 h | 24 h |
| | | 0.5 | | >10000 | >10000 |
| 0.5 | 5 | 1 | without | >10000 | >10000 |
| | | 0.5 | | 143 | 2 |
| 0.5 | 5 | 1 | 2 d mixture | 80 | 36 |
| 0.5 | 3 | 0.5 | without | >10000 | >10000 |
| | | | 2 d mixture | | 6 |
| 0.3 | 1 | 1 | without | | >10000 |
| | | | | | 2 |

### Example 27

### Prevention of the Transmission of MRSA in Skin Contacts

Methodical Approach:

For these experiments we used mouse tails from mice maintained under sterile conditions. In order to exclude any foreign source of contamination, the tails were placed into 70% alcohol for 5 minutes before the beginning of the experiments and were then dried under the laminar box.

The donor mouse tails were contaminated by placing them into a diluted MRSA-culture (Northern German strain, MF-standard 0.5 or 0.3) for 30 s up to 4 min, followed by an incubation period of 24 h at 37° C. In smear tests using these donors, germ numbers of >100,000 were detected.

The test substances were rubbed in twice a day into the acceptor tails.

The contamination of the acceptors was accomplished via skin contact with the donors for 30 s to 1 min on the shaker at 600 U/min. 2 h or 24 h after the contamination, the acceptors were smoothed down on blood-Müller-Hinton agar plates. After an incubation period of 24 h at 37° C., the colonies on the agar plates were counted.

Results:

When smoothing down the acceptors immediately after the skin contact, the plates are completely colonized (germ number>10,000). This equally applies to pre-treated acceptors and to the controls without pre-treatment, i.e. the acceptors show contamination. After 2 h or 24 h however, only low germ numbers can be detected in the pre-treated acceptors, whereas the germ numbers in the untreated controls are invariably high.

### LEGEND TO THE FIGURES

FIG. 1: Effect of micro- and nanoparticles from the marine biomass B30 (prepared according to examples 1 and 2) in comparison to the synergistic combination of B30 and vitamin C.

FIG. 2: Detection of the interruption of infective pathways simulated in the donor-acceptor model, by means of the inventive preparation according to examples 1 and 2.

FIG. 3: Testing of micro- and nanoparticles “biomass B30/vitamin C”, prepared according to the inventive preparation according to examples 1, 2, 6 in the potbelly pig.

FIG. 4: Effect of micro- and nanoparticles produced according to example 6.

FIG. 5: Detection of the interruption of infective pathways simulated in the donor-acceptor model, by means of the inventive preparation according to example 6.

FIG. 6: Particle size distribution of micro- and nanoparticles produced according to example 3.

FIG. 7: Influence of aggregates of green algal with phyllosilicates on the growth of amnion epithelium cells.

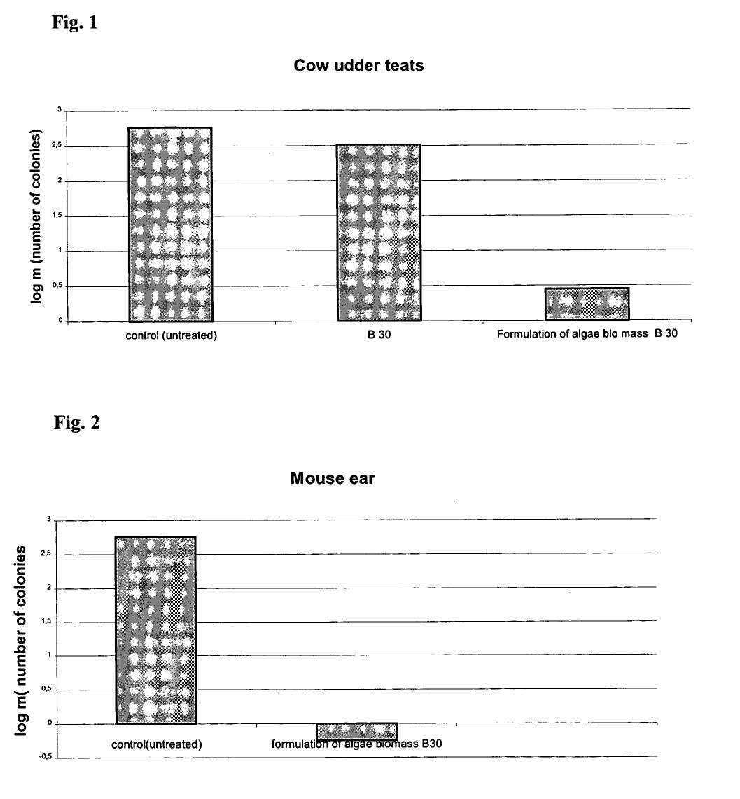

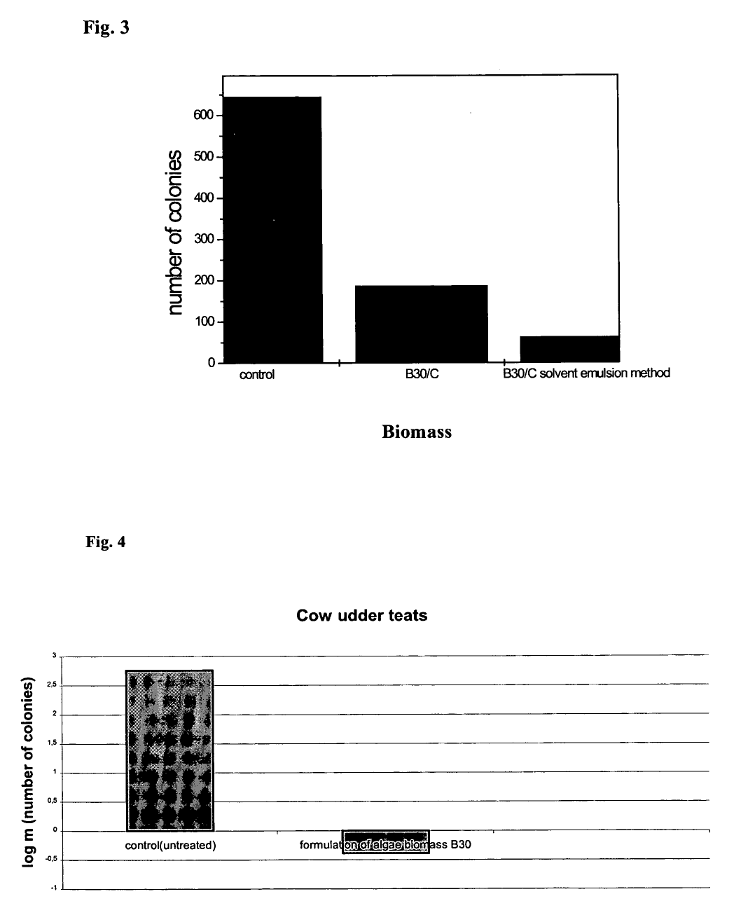

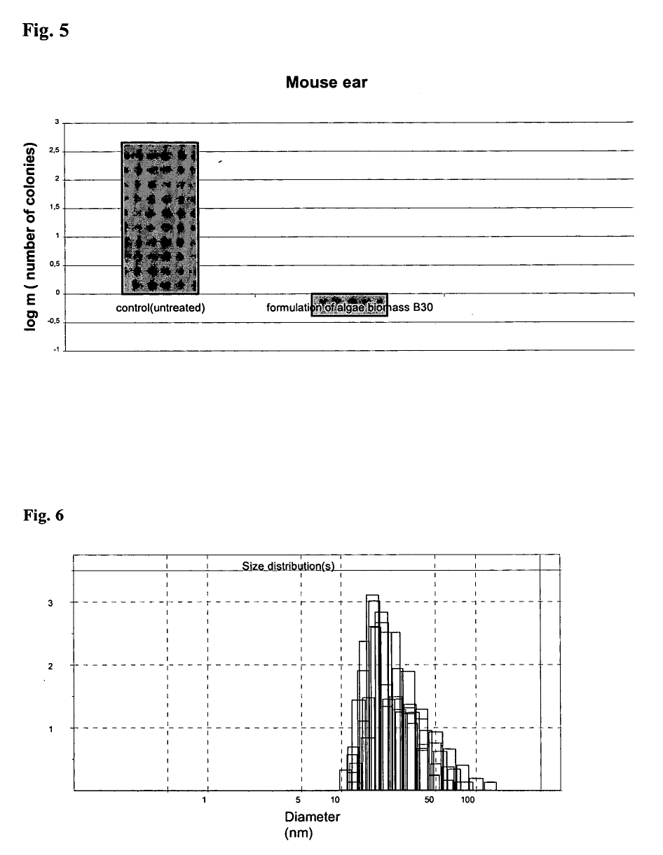

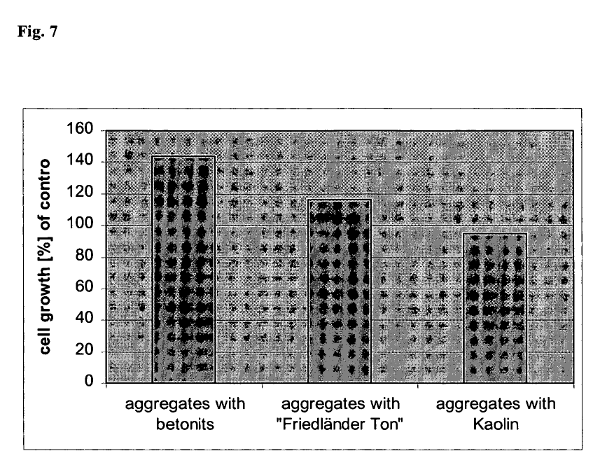

1. Pharmaceutically or cosmetically active agents, obtained by the conversion of biomasses consisting of lipid-containing marine organisms into micro- and nanoparticles.

2. Agents according to claim 1, characterised in that they have a mean diameter of 10 nm-10 μm.

3. Agents according to claim 1 or 2, characterised in that they additionally contain one or more pharmaceutical or cosmetic active substances.

4. Agents according to claim 1 or 2, characterised in that they additionally contain one or more mineral substances and/or radical scavengers and/or dietary supplements and/or vitamins, in particular vitamin C.

5. Agents according to claim 1 or 2, characterised in that they additionally contain one or more clay minerals (phyllosilicates), in particular bentonite with a diameter<2 μm.

6. Agents according to claim 3, characterised in that they contain
a) Xanthones or their derivatives and/or
b) Ubiquinones with a chain length of n=1 to n=15 and/or
c) Inorganic thiocyanates and/or
d) Hydrothiocyanates of organic bases and/or
e) Trihydroxybenzaldehyde or its derivatives and/or
f) DNA
as active substances.

7. Agent according to claim 1 or 2, characterised in that they contain norlichexanthone.

8. Agents according to one of the claim 1 or 2, characterised in that they additionally contain one or more dispersion-stabilizing substances.

9. Agents according to 1 or 2, characterised in that as lipid-containing marine organisms
a) Cyanobacteria from the class Oscillatoriales, in particular the strains SPH 03, SPH 04, SPH 05, SPH 06, SPH 09, SPH 10, SPH 11, SPH 12, SPH 13, SPH 14, SPH 20, SPH 21, SPH 22, SPH 23, SPH 25, SPH 26, SPH 29, SPH 32, SPH 34, SPH 37 and/or
b) the class Nostocales, in particular the strains SPH 18, SPH 20, SPH 27, SPH 28, SPH 38 and/or
c) the class Chroococcales, in particular the strains SPH 07a, SPH 07b, SPH 08, SPH 14, SPH 16, SPH 17, SPH 24, SPH 33, SPH 36, SPH 39, SPH 40, SPH 43 and/or
d) the class Stigonematales and/or
e) Macroalgae from the genera Asparagopsis, Cystoseira, Codium, Dictyota, Dictyopteris, Enteromorpha, Fucus, Gelidium, Gracilaria, Gracilariopsis, Halopteris, Hypoglossum, Laurencia, Plocamium, Polyneura, Sargassum, Solieria, Ulva and/or
f) Thraustochytrids from the genera Schizochytrium and Thraustochytrium and/or
g) Marine bacteria from the genera Photobacterium, Shewanella and Colwellia
are employed.

10. Agents according to claim 1 or 2, characterised in that as lipid-containing marine organisms cultivated lipid-containing marine organisms, in particular lipid-containing marine organisms cultivated in the presence of clay minerals, are employed.

11. Method for the production of pharmaceutically or cosmetically active agents according to claim 1 or 2, characterised in that biomasses of lipid-containing marine organisms are converted by homogenisation or emulsification into micro- and nanoparticles with a diameter of 10 nm-10 μm.

12. Method according to claim 11, characterised by the following steps:
Heating the marine microorganisms until the liquefaction of the fatty acids contained therein
Optionally adding one or more active substances or additives
Mixing the biomass or the charged biomass with a surfactant-water mixture heated to a temperature above the fatty acids' melting points and unification of the two phases
Preparation of a pre-suspension
High pressure homogenisation in one or more homogenisation cycles

13. Method according to claim 12, characterised in that the heating of the microorganisms and of the surfactant-water mixture is omitted and that the active substances are adsorbed at room temperature at the lipid-containing marine microorganisms or are dispersed under the addition of a little quantity of water.

14. Method according to claim 11, characterised by the following steps:
Suspending the marine microorganisms and optionally the additives in an organic solvent and pre-dispersing this mixture
High pressure homogenisation and subsequent spray drying or lyophilization
Redispersion in an aqueous surfactant solution
Again dispersion and high pressure homogenisation in one or more homogenisation cycles

15. Method according to claim 11, characterised by the following steps:
Formation of an emulsion of water and biomass and optionally with the additives
Dissolving the emulsion in an appropriate organic solvent
Adding a water-soluble co-surfactant and pre-dispersing
High pressure homogenisation and removal of the solvent

16. Use of biomasses of lipid-containing marine organisms as a carrier for active substances.

17. Use of the biomasses of lipid-containing marine organisms in the form of micro- and nanoparticles according to claim 1 as pharmaceutically or cosmetically active agents.

18. Use of biomasses of lipid-containing marine organisms in the form of micro- and nanoparticles according to claim 1 as foodstuff additives.

19. Use of biomasses of lipid-containing marine organisms in the form of micro- and nanoparticles according to claim 1 for the production of cosmetics or pharmaceuticals or foodstuffs.

20. Use according to any one of claims 17 to 19 in combination with other cosmetics or pharmaceuticals.

21. Use according to any one of claims 17 to 19 for gene transfer.

22. Use of biomasses of lipid-containing marine organisms in the form of micro- and nanoparticles according to claim 1 for preventing the binding of nosocomially important air-spread germs to receptors on the skin or tissues and/or their growth on the skin or tissues.

23. Use according to claim 22 for the improvement of the natural barrier function of the skin and/or for modifying the skin milieu.

24. Use of biomasses of lipid-containing marine organisms in the form of micro- and nanoparticles according to claim 1 for the prophylaxis of nosocomial infections.

25. Use according to any one of claims 22 to 24 for inhibiting multiresistant Staphylococcus aureus strains, in particular methicilline-resistant strains of S. aureus (MRSA).

26. Use according to any one of claims 22 to 24 for cleaning up skin being contaminated with MRSA.

27. Use according to any one of claims 22 to 24 for the skin care after the decolonization by means of bactericidal agents.

28. Use according to any one of claims 22 to 24 in combination with xanthone derivatives of the formula 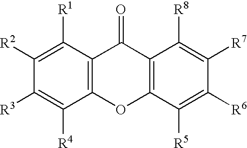
wherein R1-R8 can be selected from the substituents listed in table 1.

29. Use according to any one of claims 22 to 24 in combination with vitamins, in particular with vitamin C.

30. Use according to any one of claims 16 to 19 as a carrier for antibiotics.

31. Use according to any one of claims 17 to 19 for the dosed release of antimicrobial active substances and for simultaneous immunostimulation.

32. Use according to any one of claims 17 to 19 in slow-release systems for the prevention of implant-associated infections.

33. Use according to any one of claims 17 to 19 for the stimulation of leucocytes or for the activation of the reticuloendothelial system.

34. Use according to any one of claims 17 to 19 for the impregnation of textile and/or materials produced on a cellulose basis or as covering materials for wound treatment.

35. Use according to any one of claims 17 to 19 in the form of oils, sprays, and ointments.

36. Use according to any one of claims 17 to 19 for the acceleration of cell growth.

37. Use according to any one of claims 17 to 19 for the goal-directed substitution of deficiency syndromes.
